(19) United States
(12) Patent Application Publication
(10) Pub. No.: US 2021/0212394 A1
(43) Pub. Date: Jul. 15, 2021

(54) PATIENT WEAR WITH TREATMENT-ASSIST FEATURES

(71) Applicant: Easy Care International Co., LTD, Changhua City (TW)

(72) Inventor: Su-Lien Wu, Changhua City (TW)

(21) Appl. No.: 16/741,671

(22) Filed: Jan. 13, 2020

(51) Int. Cl. A41D 13/12; A41D 27/20; A61M 25/02

(52) U.S. Cl. CPC A41D 13/1245; A41D 27/20; A61M 2025/0206; A41D 2300/32; A61M 25/02

(57) ABSTRACT

A patient wear with treatment-assist features has a neckline, two shoulder region, two sleeves and a body region. A two-way zipper is disposed at a center position from the neckline through the body region, each sleeve has a strip opening configured to provide access for an injection, an IV tube, or medical treatments. Each strip opening is provided with a plurality of first hook and loop fasteners. Each shoulder region has a first zipper extending from the shoulder region to the body region and is configured to accept tubes for chemotherapy or dialysis. The body region has a waist region opening respectively on both sides of the two-way zipper. The waist region opening is disposed at a position corresponding to a human waist area, provided with a second hook and loop fastener, and configured for a ureter or a colostomy tube.

### BACKGROUND OF INVENTION

### Field of Invention

The present invention relates to patient, and more particularly to a patient wear with treatment-assist features

### Description of the Related Art

In typical medical institutions, the patient clothing is mostly thin, loose, easy to open and without buttons design, which mainly serves the convenience of medical care, but without warmth and privacy. Furthermore, patients with a IV drip, nasogastric tube, respirator intubation, or other medical or life-supporting tubes need to use the patient wear with better protection, because some patients may grab or remove the tubing with or without intention. The above situation is even more likely to occur with patients with mental illness. The conventional method is to use straps to directly tie the two arms of the patient to the two sides of the bed, or use a plate to fix the palms flatly on it to limit the patient's arms and palms activities. However, this method might not only cause the patients' hands to suffer poor blood circulation, but also increase the patient's discomfort.

Therefore, it is desirable to provide patient wear with treatment-assist features to mitigate and/or obviate the aforementioned problems.

### SUMMARY OF THE INVENTION

An objective of present invention is to provide patient wear with treatment-assist features, which is capable of improving the above-mention problems.

In order to achieve the above mentioned objective, a patient wear with treatment-assist features has a neckline, two shoulder region, two sleeves and a body region. A two-way zipper is disposed at a center position from the neckline through the body region, each sleeve has a strip opening configured to provide access for an injection, an IV tube, or medical treatments. Each strip opening is provided with a plurality of first hook and loop fasteners. Each shoulder region has a first zipper extending from the shoulder region to the body region and is configured to accept tubes for chemotherapy or dialysis. The body region has a waist region opening respectively on both sides of the two-way zipper. The waist region opening is disposed at a position corresponding to a human waist area, provided with a second hook and loop fastener, and configured for a ureter or a colostomy tube.

Other objects, advantages, and novel features of invention will become more apparent from the following detailed description when taken in conjunction with the accompanying drawings.

### BRIEF DESCRIPTION OF DRAWINGS

FIG. 1 is a schematic drawing of the patient wear according to a preferred embodiment of the present invention.

FIG. 2 is a schematic drawing of the use of the patient wear according to the preferred embodiment of the present invention.

FIG. 3 is a partial enlarged drawing of the neckline of the patient wear according to the preferred embodiment of the present invention.

FIG. 4 is a schematic diagram of the use of the sleeve according to the preferred embodiment of the present invention.

FIG. 5 is another schematic diagram of the use of the sleeve according to the preferred embodiment of the present invention.

FIG. 6 is a drawing of the position of the glove according to the preferred embodiment of the present invention.

FIG. 7 is a schematic diagram of the use of the glove according to the preferred embodiment of the present invention.

FIG. 8 is a sectional view of the glove according to the preferred embodiment of the present invention.

FIG. 9 is another schematic diagram of the use of the glove according to the preferred embodiment of the present invention.

FIG. 10 is another sectional view of the glove according to the preferred embodiment of the present invention.

### DETAILED DESCRIPTION OF THE PREFERRED EMBODIMENT

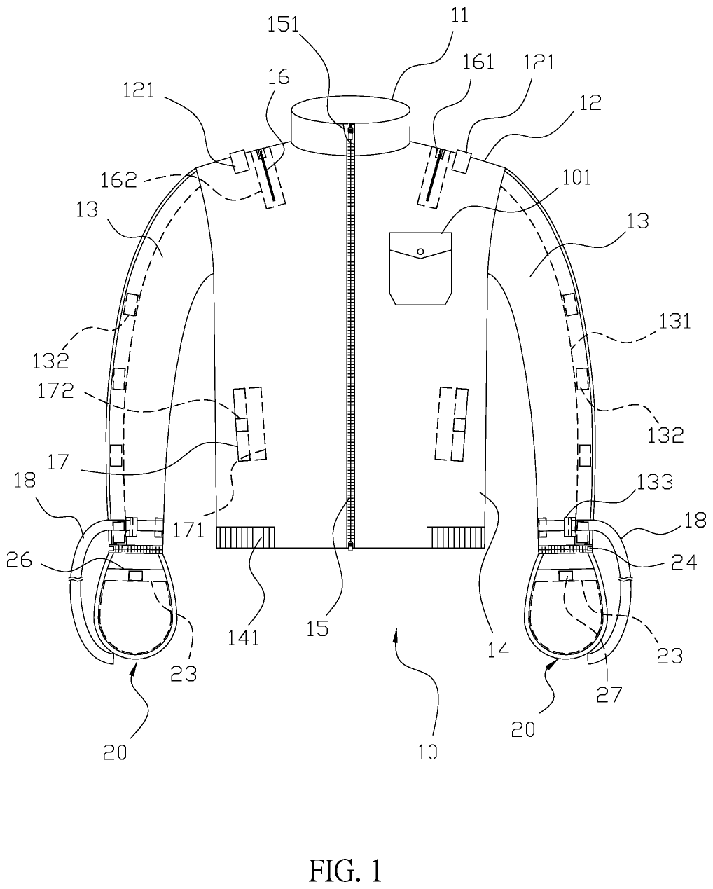

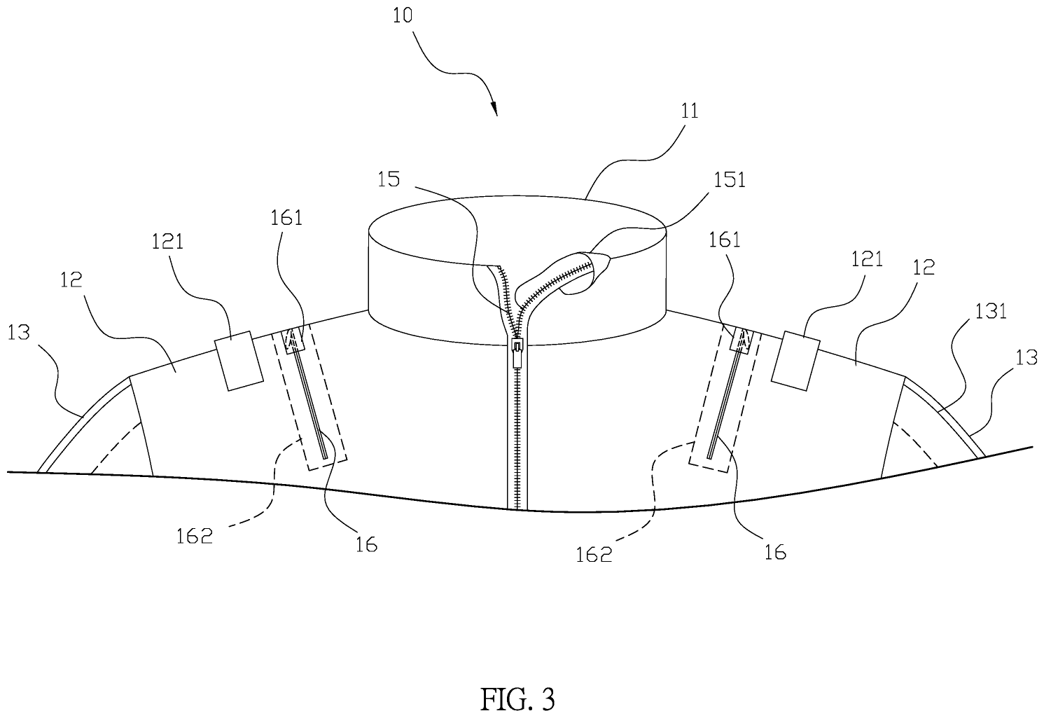

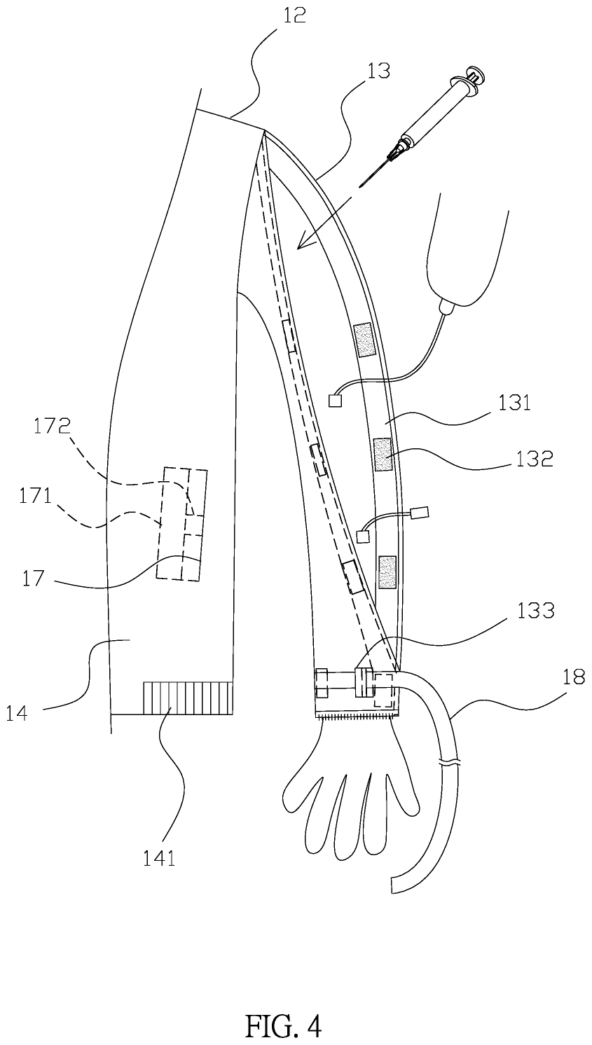

First, please refer to FIGS. 1-5. A patient wear with treatment-assist features comprises: a patient wear 10 and two gloves 20. The patient wear 10 has a neckline 11, two shoulder region 12, two sleeve 13 and a body region 14. A two-way zipper 15 is disposed at a center position from the neckline 11 through the body region 14 on the patient wear 10. The two-way zipper 15 allows a patient to put on or take off the patient wear 10, also provides two-way accesses to local torso regions such as the chest region or the abdominal region. The patient wear 10 further has a front pocket 101 on a front side for storing personal items or identification. The body region 14 further has a tightening portion 141 at a lower end portion 141, for more fitted design and better thermal effect. Each shoulder region 12 of the patient wear 10 is further provided with a secured layer 121 for securing the oxygen tube. Each sleeve 13 has a strip opening 131 configured to provide access for an injection, an IV tube, or medical treatments, and each strip opening 131 is opening provided with a plurality of first hook and loop fasteners 132. Each shoulder region 12 has a first zipper 16 extending from the shoulder region 12 to the body region 14 and having a length between 10 cm-15 cm. The first zipper 16 is configured to accept tubes for chemotherapy or dialysis. Furthermore, the two-way zipper 15 at the neckline 11 and the first zipper 16 at the shoulder regions 12 are provided with a protecting layer 151, 161, and the protecting layer 151, 161 are configured to cover the two-way zipper 15 and the first zippers 16. Moreover, the body region 14 has a waist region opening 17 respectively on both sides of the two-way zipper 15, and an inner layer 162, 171 are respectively disposed under the first zipper 16 and the waist region opening 17. The waist region opening 17 is disposed at a position corresponding to a human waist area, provided with a second hook and loop fastener 172, and configured for a ureter or a colostomy tube. In summary, when a patient wears the patient wear 10, the two-way zipper 15, the strip opening 131, the first zipper 16 and the waist region opening 17 provide direct access to different local areas, which also has the functions of rapid operation of medical methods, high comfort, high privacy and high warmth, so as to improve the use of medical care.

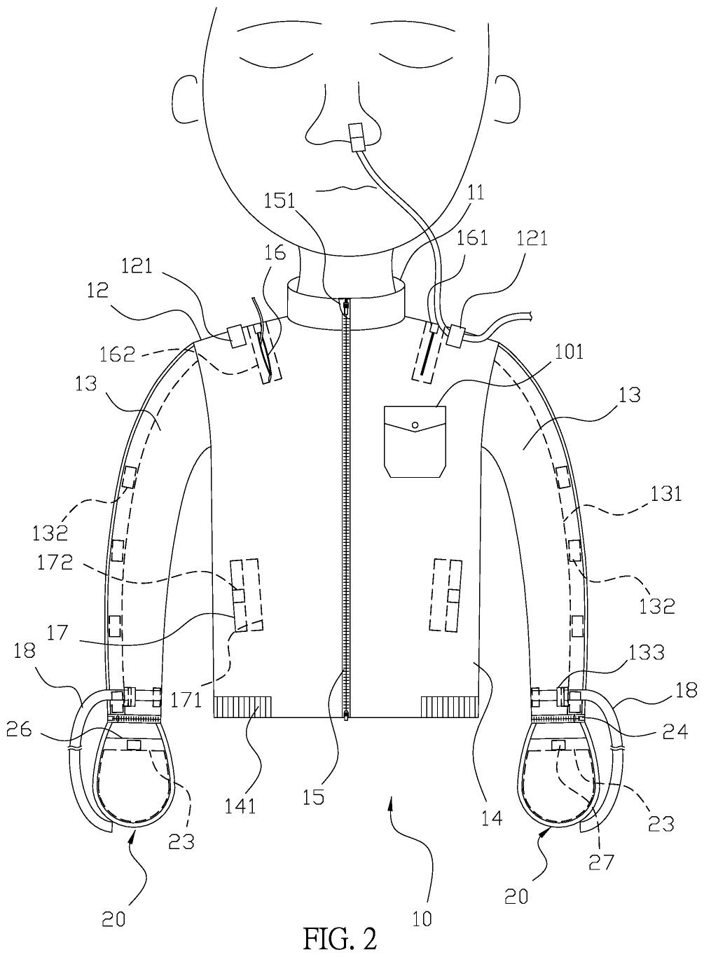

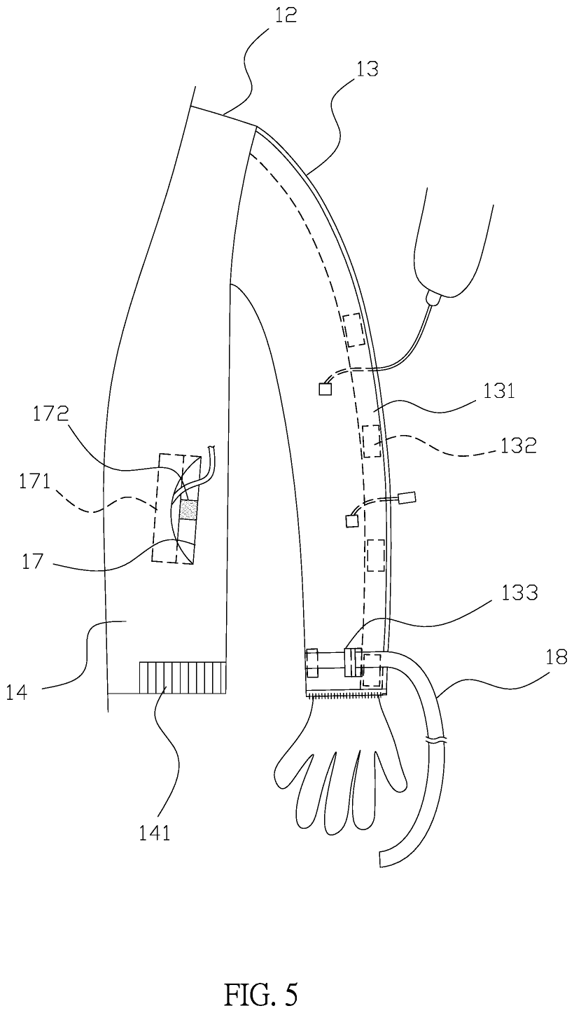

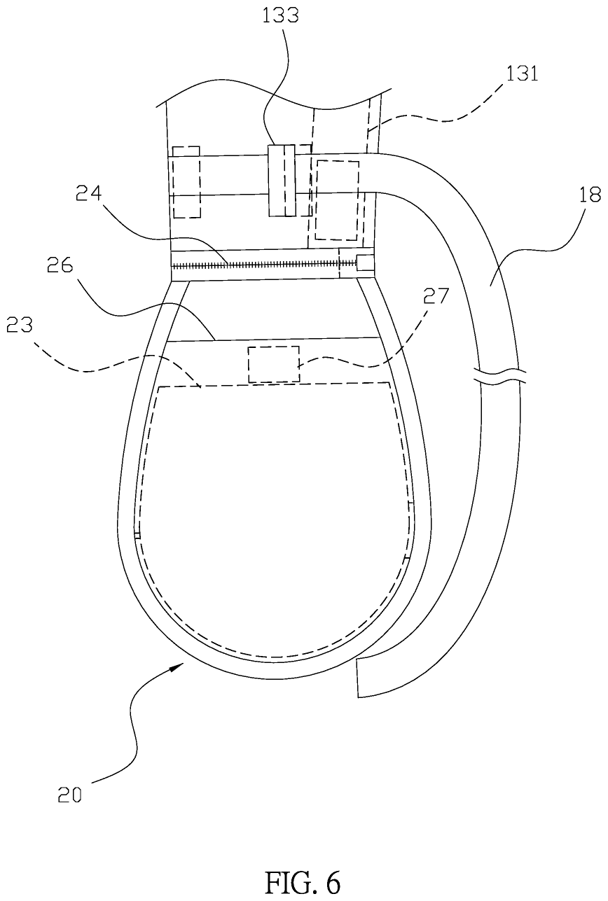

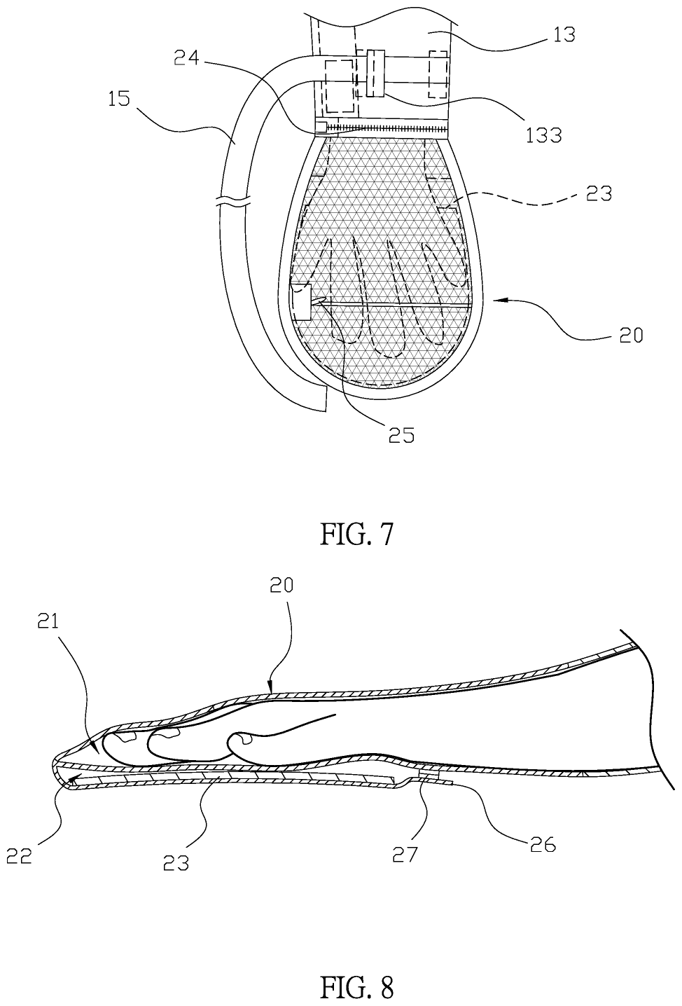

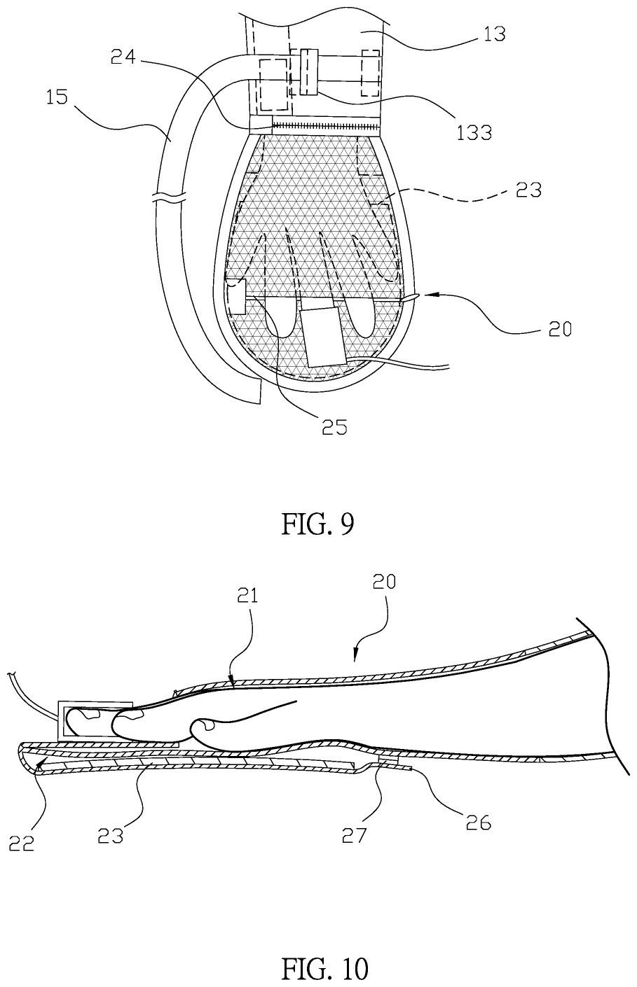

Please refer to FIG. 6 to FIG. 10 with FIG. 2. An end of each sleeve 13 is connected to a glove 20, and each glove 20 has a first pocket 21 on a front side and a second pocket 22 on a back side. The first pocket 21 is larger than the second pocket 22, the first pocket 21 and the second pocket 22 are overlapped with each other, and the second pocket 22 is provided with a bent hard plate 23. The first pocket 21 of each glove 20 is attached onto the sleeve 13 via a second zipper 24, and the first pocket 21 of each glove 29 further has a third zipper 25 disposed away from the sleeve 13 and configured to allow exposure of fingers. The second pocket 22 of each glove 20 further has a pocket opening 26 towards to the sleeve 13, and the pocket opening 26 is provided with a third hook and loop fastener 27. Each sleeve 13 further comprises a plurality of rings 133 adjacent to the glove 20, and each ring 133 is attached with a girdle 18 for straining patient's arms.

With the above structure, when the patient wears the patient wear 10, puts his or her arms in the sleeve 13, and places the hand in the first pocket 21 of the glove 20, so that the hard plate 23 is at the palm position. The bent hard plate 23 is used to improve the fit. Then, the two-way zipper 15 of the patient wear 10 can be pulled up to complete the wearing of the patient wear 10. The glove 20 provides a covering of the palm, and the hard plate 23 limits the movement range of the palm. Since the hard plate 23 only restricts one side of the palm, the back of the palm is covered by a soft covering, which can be meshed to improve its ventilation and thus the glove 20 has both its protective effect and comfort, thereby achieving the use of medical care.

Although the present invention has been explained in relation to its preferred embodiment, it is to be understood that many other possible modifications and variations can be made without departing from the spirit and scope of invention as hereinafter claimed.

What is claimed is:

1. A patient wear with treatment-assist features, comprising:
a neckline, two shoulder region, two sleeves and a body region; a two-way zipper disposed at a center position from the neckline through the body region, each sleeve having a strip opening configured to provide access for an injection, an IV tube, or medical treatments, each strip opening provided with a plurality of first hook and loop fasteners; each shoulder region having a first zipper extending from the shoulder region to the body region and configured to accept tubes for chemotherapy or dialysis; the body region having a waist region opening respectively on both sides of the two-way zipper, and the waist region opening disposed at a position corresponding to a human waist area, provided with a second hook and loop fastener, and configured for a ureter or a colostomy tube.

2. The patient wear with treatment-assist features as claimed in claim 1, wherein an end of each sleeve is connected to a glove, each glove having a first pocket on a front side and a second pocket on a back side, the first pocket being larger than the second pocket, the first pocket and the second pocket overlapped with each other, and the second pocket is provided with a bent hard plate.

3. The patient wear with treatment-assist features as claimed in claim 2, wherein the first pocket of each glove is attached onto the sleeve via a second zipper, and the first pocket of each glove further has a third zipper disposed away from the sleeve and configured to allow exposure of fingers.

4. The patient wear with treatment-assist features as claimed in claim 2 wherein the second pocket of each glove further has a pocket opening towards to the sleeve, and the pocket opening is provided with a third hook and loop fastener.

5. The patient wear with treatment-assist features as claimed in claim 2, wherein each sleeve further comprises a plurality of rings adjacent to the glove, and each ring is attached with a girdle.

6. The patient wear with treatment-assist features as claimed in claim 1, wherein the two-way zipper at the neckline region and the first zippers at the shoulder regions are provided with a protecting layer, and the protecting layers are configured to cover the two-way zipper and the first zippers.

7. The patient wear with treatment-assist features as claimed in claim 1, wherein the patient wear further has an inner layer under the first zipper and the waist region opening.

8. The patient wear with treatment-assist features as claimed in claim 1, wherein a length of the first zipper is between 10 cm-15 cm.

9. The patient wear with treatment-assist features as claimed in claim 1, wherein the body region further has a tightening portion at a lower end.

10. The patient wear with treatment-assist features as claimed in claim 1, wherein each shoulder region of the patient wear is further provided with a secured layer.
